(19) United States
(12) Patent Application Publication
(10) Pub. No.: US 2006/0034915 A1
(43) Pub. Date: Feb. 16, 2006

(54) Dexamethasone-containing formulations for oral administration as well the process for manufacturing required therefor

(76) Inventors: Paul William Rice, Tallaght (IE); Nicholas McHardy, Baltyboys (IE)

Correspondence Address: JACOBSON HOLMAN PLLC, 400 SEVENTH STREET N.W., SUITE 600, WASHINGTON, DC, 20004, US

(21) Appl. No.: 11/207,749

(22) Filed: Aug. 22, 2005

Related U.S. Application Data
(63) Continuation of application No. PCT/IE04/00024, filed on Feb. 19, 2004.
(60) Provisional application No. 60/448,174, filed on Feb. 20, 2003.

(51) Int. Cl. A61K 31/573; B27N 3/00; A61K 9/20

(52) U.S. Cl. 424/464; 514/179; 264/109

(57) ABSTRACT

A formulation of dexamethasone is prepared by preparing a dilute aqueous suspension of dexamethasone in a viscosity increasing excipient such as starch. The suspension is sprayed onto base ingredients and the resultant mixture is dried to provide a granulation of uniform potency.

This is a continuation of PCT/IE04/000024 filed Feb. 19, 2004 and published in English, claiming benefit of U.S. provisional application No. 60/448,174, filed Feb. 20, 2003.

### FIELD OF THE INVENTION

This invention relates to a method for formulating the corticosteriod dexamethasone into a granulation.

### BACKGROUND OF THE INVENTION

The manufacture of drug formulations for pharmaceutical use can be complex in preparing a single drug formulation or a combination of drugs in a formulation for optimum efficacy. Different drugs can have very different properties and require different handling methods.

Dexamethasone is a very potent corticosteroid; it has thirty times the anti-inflammatory potency of cortisone (The Pharmacological basis of Therapeutics, Ninth edition, Goodman and Gilman, page 1466). As a result, only a small amount of drug is needed in the oral dosage form (tablet) which is given to the patient. Generally, only 0.25 mg dexamethasone is present in a 300 mg tablet (0.083%). The challenge is to uniformly disperse the small amount of drug in the tablet granulation to provide uniformity of dose in the compressed tablets.

Historically, dexamethasone has been incorporated into the tablet granulation either by direct compression or wet granulation.

Direct compression techniques use ordered mixing to gradually mix the dexamethasone with increasing amounts of excipient. While a uniform mix may result in the blender, segregation may occur as the granulation is transferred into other containers or while being fed into the tablet press. This “in process” segregation can result in non uniform tablets.

Wet granulation techniques typically use solvents such as ethanol or isopropanol which dissolve the dexamethasone into a spray solution which is sprayed onto the tablet granulation. The problem with this approach is largely the environmental impact of venting the spent solvents into the environment. Solvent recovery technology exists but is very expensive. Industry has been pressed to eliminate solvents wherever possible due to the costs of solvent recovery.

WO 96/40078A describes dissolving dexamethasone and polyvinyl pryrollidone in ethanol. The solution is added drop-wise to guar gum to form granules.

JP 11130663 describes suspension of biotin in hydroxy propyl cellulose or hydroxy propyl methylcellulose and water. The suspension is dispersed using a wet granulation technique.

There is therefore a clear need for an improved process for preparing a pharmaceutical granulation of dexamethasone.

### STATEMENTS OF INVENTION

According to the invention there is provided a process for manufacturing a formulation of dexamethasone comprising preparing a dilute aqueous suspension of dexamethasone, spraying the suspension onto base ingredient(s), and drying the resultant mixture to provide a granulation. Preferably the dilute aqueous suspension is prepared by mixing the compound with a viscosity increasing excipient.

In one embodiment of the invention the viscosity increasing excipient is selected from any one ore more of starch, pre-gelatinised starch, hydroxypropylmethylcellulose, methycellulose or povidone. Preferably the viscosity increasing excipient is starch.

Preferably weight ratio of the viscosity increasing excipient to the pharmaceutical compound is from 12:1 to 60:1, most preferably approximately 30:1.

In one embodiment of the invention the viscosity increasing excipient is present in an amount of from 0.5% to 5.0% w/w of the tablet formulation.

In one embodiment the process comprises milling the aqueous mixture of dexamethasone and die viscosity increasing excipient to form a dexamethasone suspension.

The process may comprise suspending the viscosity increasing excipient in water and subsequently adding the dexamethasone to the suspension to form a mixture.

In one embodiment the suspension is sprayed onto the base ingredient(s) in a fluidised bed dryer.

In one embodiment the resulting granulation is compressed into a tablet. Preferably the pharmaceutical compound is present in the tablet in an amount of from 2 to 0.02% w/w of the formulation, most preferably in an amount of approximately 0.083% w/w.

The dexamethasone may be in the form of dexamethasone base.

The invention provides a granulation or powder dosage form whenever manufactured by a process of the invention.

The term granulation is taken throughout to include from very fine dusty powders to larger particle sizes such as granules.

The invention also provides a tablet or tablet dosage form whenever manufactured by a process of the invention.

Preferably the tablet comprises an amount of from 2 to 0.02% w/w of the formulation of dexamethasone and from 0.5% to 5% w/w of the formulation of a viscosity increasing excipient.

In one embodiment the viscosity increasing excipient is selected from any one or more of starch, pre-gelatinised starch, hydroxypropyl-methylcellulose or povidone, preferably the viscosity increasing excipient is starch.

### DETAILED DESCRIPTION

The present invention utilizes a viscous aqueous based spray solution to provide a uniform distribution of a small amount of dexamethasone in a large amount of granulation. After the water evaporates, dexamethasone is bound to the granulation, reducing the potential for downstream segregation.

It was surprisingly found that dexamethasone could be combined with water and a viscosity building excipient and suspended in a spray solution.

A variety of excipients may be used to provide sufficient viscosity to the spray solution. Examples of suitable excipients include starch, pregelatinised starch, hydroxypropylmethylcellusose, methylcellulose and povidone. The amount required depends on a number of factors such as the time a solution is left standing, and the degree of agitation of the mixer.

In the invention starch was used as the viscosity increasing excipient to male the spray solution comprising dexamethasone. A range of concentrations of starch paste were prepared and milled to observe the cut off point for the dexamethasone separating out. The range of 0.5% to 5.0% was investigated. The 0.5% sample showed some settling, but was readily dispersed. Even the 0.5% concentration would be acceptable when it is continuously mixed during spraying.

In the invention dexamethasone is incorporated into a very dilute paste with sufficient viscosity to prevent separation and ensure a uniform, suspended ingredient. The spray solution must be sufficiently dilute to be sprayed on the batch. The excipients providing viscosity to the aqueous solution act as “glue” and bind the dexamethasone to the granulation after the water evaporates away. The resulting granulation is uniform and the dexamethasone is bound to the granulation which minimizes segregation during subsequent product transfer.

Base ingredients may include any conventionally used carrier ingredient commonly used in the art. For example: lactose, microcrystalline cellulose, dibasic calcium phosphate, starch, dextin, maltodextrin, compressible sugar, dextrose, and calcium sulfate. The mixing sequence used for dexamethasone includes preparing the starch paste, adding dexamethasone to a portion of the starch paste to make a slurry (lumps may be present), then milling the slurry once or multiple times until it is a uniform suspension, followed by purging the mill with starch paste to remove residual dexamethasone in the mill. The milled suspension and purge are then added to the spray solution tank for spraying onto the granulation using a Glatt Fluid bed drier.

Advantageously, the starch is approximately 0.5% to 5.0% weight/weight of the batch.

Advantageously, the excipient used to provide viscosity to the spray solution is starch, pregelatinized starch, hydroxypropylmethylcellulose, methylcellulose, or povidone.

The composition may be provided in the form of a uniform granulation which may be used a powder dosage form.

Alternatively the composition may be provided in the form of a uniform granulation which can be compressed into tablets.

The invention will be more clearly understood by the following example.

### EXAMPLE

A 540 kg batch of dexamethasone 0.25 mg tablets was prepared according to the following procedure:

Composition:

| Ingredient | Quantity | |
|---|---|---|
| Dexamethasone | 0.45 | kg |
| Starch USP | 5.4 | kg |
| Water, Cold | 10.8 | kg |
| Water, Hot | 178.2 | kg |

| | |
|---|---|
| Granulation base ingredients | QS to 540 kg |

Procedure:

1. Add the starch to the cold water with stirring to make a smooth suspension.

2. Add the suspension (step 1) to the hot water with stirring.
A starch paste will quickly form.

3. Remove approximately 18 kg of starch paste and place in a suitable container.

4. Add the dexamethasone to the container and mix together to get a dispersion, there may be lumps in the dispersion.

5. Pass the dispersion through a suitable mill to break up lumps of dexamethasone and produce a smooth uniform dispersion. In this example, the mill was a Charlotte Colloid Mill, Model # SD2.

6. Pass the dispersion through the mill again to further ensure the dexamethasone is all milled.
(Repeat milling may be required to provide a uniform suspension).

7. Purge the mill with approximately 14 kg starch paste to remove residual dexamethasone.

8. Add the milled dexamethasone slurry and the purge back into the starch paste.

9. Mix until uniform in appearance.

10. Spray the dexamethasone suspension on the base ingredients in the a Glatt Fluid Bed Agglomerator/Drier, Model WSG-500.

11. Dry the granulation in the Glatt Drier to 2-3% moisture.

12. Remove the granulation from the Glatt drier and transfer to a Tote bin.

13. Assay the granulation and calculate tablet weight needed for 100% potency.

14. Using the target tablet weight, compress the granulation into tablets.

The resulting dexamethasone 0.25 mg tablets are uniform in potency. Content uniformity testing gave an average of 101.2% claim with an RSD of 0.8%.

The invention is not limited to the embodiments hereinbefore described which may be varied in detail.

1-20. (canceled)

21. A process for manufacturing a formulation of dexamethasone comprising preparing a dilute aqueous suspension of dexamethasone, spraying the suspension onto base ingredient(s), and drying the resultant mixture to provide a granulation.

22. The process as claimed in claim 21 wherein the dilute aqueous suspension is prepared by mixing dexamethasone with a viscosity increasing excipient.

23. The process as claimed in claim 22 wherein the viscosity increasing excipient is selected from any one or more of starch, pre-gelatinised starch, hydroxypropylmethylcellulose, methycellulose or povidone.

24. The process as claimed in claim 22 wherein the viscosity increasing excipient is starch.

25. The process as claimed in claim 22 wherein weight ratio of the viscosity increasing excipient to the pharmaceutical compound is from 12:1 to 60:1.

26. The process as claimed in claim 25 wherein the ratio is approximately 30:1.

27. The process as claimed in claim 22 wherein the viscosity increasing excipient is present in an amount of from 0.5% to 5.0% w/w of the tablet formulation.

28. The process as claimed in claim 22 comprising milling the aqueous mixture of dexamethasone and the viscosity increasing excipient to form a dexamethasone suspension.

29. The process as claimed in claim 22 comprising suspending the viscosity increasing excipient in water and subsequently adding the dexamethasone to the suspension to form a mixture.

30. The process as claimed in claim 21 wherein the dexamethasone suspension is sprayed onto the base ingredient(s) in a fluidised bed dryer.

31. The process as claimed in claim 21 wherein the resulting granulation is in the form of a powder dosage form.

32. The process as claimed in claim 21 wherein the resulting granulation is compressed into a tablet or tablet dosage form.

33. The process as claimed in claim 31 wherein dexamethasone is present in the granulation in an amount of from 2 to 0.02% w/w of the formulation.

34. The process as claimed in claim 31 wherein dexamethasone is present in the granulation in an amount of approximately 0.083% w/w.

35. The process as claimed in claim 21 wherein the dexamethasone is in the form of dexamethasone base.

36. The powder dosage form whenever manufactured by a process as claimed in claim 21.

37. The tablet or tablet dosage form whenever manufactured by a process as claimed in claim 21.

38. A tablet comprising an amount of from 2 to 0.02% w/w of the formulation of dexamethasone and from 0.5% to 5% w/w of the formulation of a viscosity increasing excipient.

39. The tablet as claimed in claim 38 wherein the viscosity increasing excipient is selected from any one or more of starch, pre-gelatinised starch, hydroxypropyl-methylcellulose or povidone.

40. The tablet as claimed in claim 38 wherein the viscosity increasing excipient is starch.
